(19) United States
(12) Patent Application Publication
(10) Pub. No.: US 2006/0281154 A1
(43) Pub. Date: Dec. 14, 2006

(54) Enzymatic depolymerisation of carboxymethylcellulose in hydroalcoholic solutions

(76) Inventors: Giuseppe Molteni, Varese (IT); Cristina Formantici, lerago con Orago (IT); Cristina Demarchi, Berlinzago (IT); Yves Galante, Arese (IT); Giuseppe Libassi, Gavinata (IT)

Correspondence Address: PAUL S MADAN;MADAN, MOSSMAN & SRIRAM, PC, 2603 AUGUSTA, SUITE 700, HOUSTON, TX, 77057-1130, US

(21) Appl. No.: 10/564,741

(22) PCT Filed: Jul. 14, 2004
(86) PCT No.: PCT/EP04/51491

§ 371 Date: May 31, 2006

(30) Foreign Application Priority Data
Jul. 17, 2003 (IT) VA2003A000025

(51) Int. Cl. C12P 19/04; A61K 31/716

(52) U.S. Cl. 435/101; 514/57

(57) ABSTRACT

The present invention refers to the procedure for the preparation of aqueous concentrated solutions of low viscosity carboxymethylcellulose by enzymatic depolymerisation of medium viscosity carboxymethylcellulose in the form of dispersed powder in a hydro-alcoholic heterogeneous medium, and to such aqueous concentrated solutions which have Brookfield viscosity, at 20° C. and 20 rpm, comprised between 2000 and 5000 mPa*s. Preferred alcohol-water biphasic systems have from 40% to 50% of alcohol (e.g., ethanol or isopropanol). The aqueous solutions of carboxymethylcellulose produced are stable over time, and their viscosity varies no more than 10% in 3 months.

### TECHNICAL FIELD

This invention refers to the procedure for the preparation of aqueous concentrated solutions of low viscosity carboxymethylcellulose by enzymatic depolymerisation of medium viscosity carboxymethylcellulose in the form of dispersed powder in a hydro-alcoholic heterogeneous medium, and to such aqueous concentrated solutions.

The aqueous solutions of carboxymethylcellulose obtained with the procedure of the invention have low viscosity, can be easily handled with the conventional industrial pumping devices, have high content of carboxymethylcellulose, are stable and ready-to-use.

### BACKGROUND ART

Low viscosity carboxymethylcellulose is employed in various industrial fields, where excellent filming properties and/or adhesion are required and highly concentrated carboxymethylcellulose solutions are needed.

The rheological properties of carboxymethylcellulose are strongly influenced by its molecular weight; the degree of polymerisation of carboxymethylcellulose (DP) is an index of molecular weight and is therefore directly related to the Theological behaviour of carboxymethylcellulose solutions, thus to viscosity.

High DPs, which are characteristic of high viscosity carboxymethylcellulose preparations, are obtained using as starting material the so called cellulose linters (cotton cellulose); medium DPs, typical of medium viscosity carboxymethylcelluloses, are obtained using wood cellulose as starting material.

On the other hand, low or very low viscosity and low DP carboxymethylcellulose are obtained by depolymerising medium or high viscosity carboxymethylcellulose.

In the present text, with the expression “low viscosity carboxymethylcellulose” we mean carboxymethylcellulose whose aqueous solutions at 20-40% by weight (wt %) have BrookField viscosity from 2000 to 5000 mPa*s, at 20° C. and 20 rpm; with the expression “medium viscosity carboxymethylcellulose” we mean carboxymethylcellulose whose aqueous solutions at 4 wt % have Brookfield viscosity from 20 to 1000 mPa*s, at 20° C. and 20 rpm.

Many chemical, physical and enzymatic methods useful for the depolymerisation of carboxymethylcellulose are known; we cite, by way of example, those reported in:
EP 382577 A, where enzymatically hydrolysed cellulose derivatives are described:
EP 465992 A, where a procedure for the depolymerisation of cellulose ethers with hydrogen peroxide in water is described;
EP 708113 A, where the obtainment of low molecular weight cellulose ethers by irradiation is described;
GB 2281073, where the procedure for obtaining solutions of low viscosity carboxymethylcellulose from the dissolution of solid mixtures of carboxymethylcellulose and enzymes is described.

Numerous problems are encountered when the known methods of depolymerisation are applied:
discolouring (yellowing) of depolymerised carboxymethylcellulose when chemical agents are used, together with the formation of several by-products;
the problem of stopping enzymatic activity when enzymes (cellulases) are used, leading frequently to very low viscosity carboxymethylcellulose, (that is to carboxymethylcellulose having Brookfield viscosity lower than 50 mPa*s at 20° C., 20 rpm and 20 wt %) or anyway leading to solutions whose viscosity is unstable over time;
when carboxymethylcellulose is depolymerised in aqueous solution with enzymes, the problem of eliminating large amounts of water necessary to solubilize carboxymethylcellulose at the beginning of the process;
when depolymerisation is carried on by irradiation, the problem of the complexity and the high cost of the equipment needed.

For all the reasons stated above, a process for the preparation of concentrated aqueous solutions of low viscosity carboxymethylcellulose stable over time, with constant colour, ready to use, having viscosity not lower than 50 mPa*s (at 20° C., 20 rpm and 20 wt %) and easily achievable on a large industrial scale is still not known in the art.

### DISCLOSURE OF INVENTION

It has now surprisingly been found that it is possible to depolymerise medium viscosity carboxymethylcellulose by an enzymatic treatment in a hydro-alcoholic environment, in which carboxymethylcellulose is not dissolved but present in the form of solid dispersion (slurry), and that it is possible: to stop the enzymatic activity when the depolymerisation is completed by simple thermal treatment at alkaline pH of the mixture, to completely eliminate the alcohol by distillation and to obtain a concentrated, aqueous solution of carboxymethylcellulose having controlled, low viscosity, ready-to-use and stable over time.

The process according to the invention can be accomplished by means of normal industrial reactors; it avoids the need for the elimination of large quantities of water (with considerable saving in operating time and energy) and it preserves the product from excessive thermal stress.

The enzymatic depolymerisation of carboxymethylcellulose has been also studied in academic laboratories and described in many publications, by way of example in: Yu Cao et al., Carbohydrate Research, 337 (2002), 1291-1296; Siddiqui K. S. et al., in Enzyme and Microbial Technol., 27 (2000) 467-474; Kumakura M. et al., in Z. Naturforsch., 38c, (1983) 79-82.

However, no publication describes the enzymatic depolymerisation in heterogeneous two phase system of carboxymethylcellulose in the form of a solid dispersion in hydro-alcoholic medium.

Furthermore, the fact that enzymes, being proteins, are inactive or almost inactive or denatured in strongly alcoholic medium is a common general knowledge and belief.

It is a fundamental object of the present invention a process for the preparation of an aqueous solution of carboxymetylcellulose containing from 20 to 40 wt % of carboxymethylcellulose, having Brookfield viscosity at 20° C. and 20 rpm from 2000 and 5000 mPa*s, characterised by the fact that it comprises the following steps:

a. from 20 to 30 pbw (parts by weight) of medium viscosity carboxymethylcellulose are dispersed in 100 pbw of a mixture of water and alcohol containing from 30 to 60 wt %, preferably from 40 to 50 wt %, of alcohol;

b. the obtained dispersion is heated at a temperature of 35-55° C., 0.5 to 10 pbw (each 100 pbw of carboxymethylcellulose) of a cellulose preparation are added, and the mixture is stirred at this temperature for 60-200 minutes;

c. the alcohol is removed by distillation;

d. the cellulose preparation is deactivated by alkalinising at a pH from 11 to 13 and heating at 60-70° C. for 20-120 minutes;

e. after cooling at 40-55° C., from 1 to 5 pbw (each 100 pbw of carboxymethylcellulose) of a 30-35 wt % aqueous solution of hydrogen peroxide are added, the mixture is stirred at 55-70° C. for 15-45 minutes.

Optionally, the final carboxymethylcellulose concentration is adjusted by adding water.

Preferably, if, at the end of step e. of the process of the invention, more than 100 ppm of hydrogen peroxide are present in the aqueous solution, a catalase (an enzyme which dismutates hydrogen peroxide into oxygen and water) is added, stirring for 10-20 minutes.

The carboxymethylcellulose normally used for the realisation of the present invention has a DS (Degree of substitution) comprised between 0.5 and 1.0, preferably between 0.6 and 0.8; preferably its Brookfield viscosity, at 4 wt %, 20 rpm and 20° C., is from 20 to 500 mP*s.

The alcohols which can be used for the realisation of the invention are those completely miscible with water in all proportions: particularly preferred are ethanol and isopropanol.

Preferably, the pH of the dispersion from step a. is adjusted to 5-7 by adding acetic acid or NaOH.

Among the preparations of cellulase that can be used in the process of the invention are the commercially available preparation of cellulase containing natural cellulase complexes having endoglucanase activity (EG-I, EG-II, EGIII), exoglucanase activity (CBH-I and CBH-II) and β-glucosidase activity, or, preferably, preparation of cellulase without CBH-I but EG-I and EG-II enriched, or having a single EG-III activity expressed by a cloned gene; by way of example we cite the products Indiage® Super L and Indiage® MAXL, commercialised by Genencor, Ecostone® L/900 from AB Enzymes; Denimax® 991 L, Denimax® 601L, Denimax® 399 and Denimax® Acid XCL from Novozymes, Rocksoft® ACE from Dyadic International.

The utilisable microbial sources (strains) of the preparation of cellulase are several, such as: Trichoderma, Streptomyces, Aspergillus, Humicola, Mycelophthora, Chrisosporium, Melanocarpus ecc.

Normally, alcohol removal is performed by distillation under vacuum at a temperature of 40-45° C. for periods that can vary from 4 to 12 hours.

The aqueous solutions containing from 25 to 40 wt % of carboxymethylcellulose, obtained by enzymatic depolymerisation in hydro-alcoholic heterogeneous phase from medium viscosity carboxymethylcellulose, and having Brookfield viscosity from 2000 to 5000 mPa*s, are stable and are a fundamental aspect of the present invention.

With the expression “stable aqueous solutions” we mean aqueous solutions whose Brookfield viscosity at 20° C. and 20 rpm, expressed in mPa*s, does not vary of more than by 10% when measured after three months at room temperature.

The aqueous solutions of the invention has colour characteristics which render them suitable for direct use, without the need for whitening treatments, but can be further whitened, when specific application requirements are present.

The following examples illustrate the preparation of aqueous solutions of low viscosity carboxymethylcellulose and are not intended to unduly limit the invention.

### EXAMPLE 1

In a 130 l reactor 20 Kg of Carbocel® MM3 150 (carboxymethylcellulose having DS 0.6-0.8, Brookfield viscosity 200-500 mPa*s, at 4 wt %, from Lamberti SpA) are dispersed under stirring in 80 Kg of a mixture water/isopropanol (containing 41 wt % of isopropanol).

The pH is adjusted to 6.4 with 0.16 Kg of 50 wt % NaOH and 9.1 Kg of 80 wt % acetic acid, and the mixture is heated to 40° C. 300 g of Indiage® Super L (a preparation of cellulase commercialised by Genencor International, having enzymatic activity of 2850 GTU/g) are then added.

The dispersion is stirred for 180 minutes; a mixture of water and alcohol is then removed by distillatlon under vacuum at 40-45° C. until to residual concentration of alcohol is 0.5 wt % (determined by Gas Chromatography); NaOH is added to raise the pH to 11.5 and the mixture is stirred at 67° C. for 60 minutes.

The mixture is cooled to 50° C., 0.6 Kg of a 30 wt % aqueous solution of hydrogen peroxide are added, then heated to 65° C. and stirred for 30 minutes.

300 g of Terminox® 50 Ultra (a catalase from Novozymes, DK) are added and the mixture is stirred for 10 minutes.

The solution is cooled to 30° C. and 75 g of Carbosan CD40 (biocide from Lamberti SpA) are added.

An aqueous solution of carboxymethylcellulose having a dry content of 25 wt %, Brookfield viscosity, at 20 rpm and 20° C., of 4400 mPa*s, and pH 8 is obtained; the viscosity of the solution is stable (varying less than 10% in three months).

### EXAMPLE 2

In a reactor 675 Kg of Carbocel® MB2C 150 (carboxymethylcellulose having DS 0.6-0.8, Brookfield viscosity of 20-50 mPa*s, at 4 wt %, from Lamberti SpA) are dispersed under stirring in 2320 Kg of a mixture water/isopropanol (containing 48 wt % of isopropanol).

The pH is adjusted to 6.4 with 6.75 Kg of 50 wt % NaOH and 18.5 Kg of 80 wt % acetic acid, and the mixture is heated to 40° C.

22 Kg of Indiage® Super L (a preparation of cellulase commercialised by Genencor International, having enzymatic activity of 2850 GTU/g) are added.

The dispersion is stirred for 180 minutes; a mixture of water and alcohol is removed by distillation under vacuum at 40-45° C. over a period of 5.5 hours until the residual concentration of alcohol is 0.5 wt % (determined by Gas Chromatography); NaOH is added to adjust the pH at 11.5 and the mixture is stirred at 70° C. for 45 minutes.

The mixture is cooled to 50° C., 20 Kg of a 30 wt % aqueous solution of hydrogen peroxide are added, heated to 60° C. and stirred for 20 minutes.

1000 g of Terminox® 50 Ultra (catalase from Novozymes, DK) are added and the mixture is stirred for 10 minutes more.

The solution is cooled to 30° C. and 2.5 Kg of Carbosan CD40 (biocide from Lamberti SpA) are added.

An aqueous solution of carboxymethylcellulose having a dry content of 25 wt %, Brookfield viscosity, at 20 rpm and 20° C., of 4400 mPa*s, and pH 8 is obtained; the viscosity of the solution is stable (varying less than 10% in three months).

### EXAMPLE 3

In a 8000 l reactor 1450 Kg of Carbocel® MM3 (carboxymethylcellulose having DS 0.6-0.8, Brookfield viscosity 200-500 mPa*s, at 4 wt %, from Lambert SpA) are dispersed under stirring in 5720 Kg of a mixture water/ethanol (containing 49 wt % of ethanol).

The pH is adjusted to 5.6 with 14.5 Kg of 50 wt % NaOH and 38.9 Kg of 80 wt % acetic acid, and the mixture is heated to 44° C.

118 Kg of Indiage® MAX L (a preparation of cellulases commercialised by Genencor International, having enzymatic activity on carboxymethylcellulose of 11000 IU/g) are then added.

The dispersion is stirred for 145 minutes; a mixture of water and ethanol is removed by distillation under vacuum at 40-43° C. over a period of 5.5 hours until the residual concentration of alcohol is 0.5 wt % (determined by Gas Chromatography); 80 Kg of 50 wt % NaOH are added and the mixture is stirred at 67° C. for 60 minutes.

The mixture is cooled to 60° C., 38.7 Kg of a 30 wt % aqueous solution of hydrogen peroxide are added, heated to 65° C. and stirred for 30 minutes.

The mixture is cooled to 35° C. and 15.2 Kg of Terminox® 50 Ultra (a catalase from Novozymes, DK) are added and the mixture is stirred for 20 minutes more.

An aqueous solution of carboxymethylcellulose having a dry content of 36.4 wt %, Brookfield viscosity, at 20 rpm and 20° C., of 4400 mPa*s, and pH 7.8 is obtained; the viscosity of the solution is stable (varying less than 10% in three months).

1-9. (canceled)

10. A Process for the preparation of an aqueous solution of carboxymethylcellulose containing from about 20 to about 40 wt % of carboxymethylcellulose, having a Brookfield viscosity at 20° C. and 20 rpm of from about 2000 to about 5000 mPa*s, comprising:
a. dispersing from about 20 to about 30 pbw (parts by weight) of carboxymethylcellulose having a degree of substitution of between about 0.5 and about 1.0 and whose aqueous solutions at 4 wt % have a Brookfield viscosity of from about 20 to about 1000 mPa*s, at 20° C. and 20 rpm in 100 pbw of a mixture of water and alcohol containing from about 30 to about 60 wt % of alcohol to form a dispersion;
b. heating the dispersion at a temperature of from about 35 to about 55° C. and adding from about 0.5 to about 10 pbw of a cellulase preparation for each 100 pbw of carboxymethylcellulose to the dispersion, and stirring the dispersion at this temperature for from about 60 to about 200 minutes;
c. removing the alcohol from the mixture by distillation;
d. deactivating the cellulase preparation by alkalinizing and heating the mixture at from about 60 to about 70° C. for from about 20 to about 120 minutes; and
e. cooling the mixture at from about 40 to about 55° C. and adding from about 1 to about 5 pbw of a 30 to 35 wt % aqueous solution of hydrogen peroxide per each 100 pbw of carboxymethylcellulose to the dispersion and stirring the dispersion at from about 55 to about 70° C. for from about 15 to about 45 minutes to form an aqueous solution of carboxymethylcellulose.

11. The process of claim 1, further comprising adjusting the carboxymethylcellulose concentration of the aqueous solution of carboxymethylcellulose by adding water to the aqueous solution of carboxymethylcellulose.

12. The process of claim 1, wherein the alcohol is ethanol or isopropanol.

13. The process of claim 1, wherein the carboxymethylcellulose which is dispersed in step a. has a degree of substitution between about 0.6 and about 0.8.

14. The process of claim 1, wherein the carboxymethylcellulose which is dispersed in step a. has a Brookfield viscosity at 4 wt % of between about 20 and about 500 mPa*s, at 20° C. and 20 rpm.

15. The process of claim 1, wherein the cellulase preparation of step b. is a preparation comprising natural cellulase complexes having endoglucanase activity (ES-I, EG-II, EGIII), exoglucanase activity (CBH-I and CBH-II), and β-glucosidase activity.

16. The process of claim 1, wherein the cellulase preparation of step b. is selected from the group consisting of a cellulase preparation without CBH-I but enriched in EG-I and EG-II, a cellulase preparation having a single EG-III activity expressed by a cloned gene, and mixtures thereof.

17. The process of claim 1, wherein the mixture of water and alcohol contains from 40 to 50 wt % alcohol.

18. The process of claim 1, wherein the pH of step a. is adjusted to from about 5 to about 7 using an acid or base.

19. The process of claim 18, wherein the acid is acetic acid and the base is NaOH.

20. The process of claim 1, further comprising a step f. of adding a catalase to the solution and stirring the solution.

21. The process of claim 20, further comprising adjusting the carboxymethylcellulose concentration of the aqueous solution of carboxymethylcellulose by adding water to the aqueous solution of carboxymethylcellulose.

22. The process of claim 1, wherein the distillation is a vacuum distillation.

23. The process of claim 21, wherein the distillation is a vacuum distillation.

24. The process of claim 1, where in the cellulase in the cellulase preparation is derived from microorganisms selected from the group consisting of Trichoderma, Streptomyces, Aspergillus, Humicola, Mycelophthora, Chrisosporium, Melanocarpus, and mixtures thereof.

25. The process of claim 1, wherein the aqueous solution of carboxymethylcellulose is stable.

26. An aqueous solution of carboxymethylcellulose containing from about 20 to about 40 wt % of carboxymethylcellulose, having a Brookfield viscosity at 20° C. and 20 rpm of from about 2000 to about 5000 mPa*s, prepared by a method comprising:
a. dispersing from about 20 to about 30 pbw (parts by weight) of carboxymethylcellulose having a degree of substitution of between about 0.5 and about 1.0 and whose aqueous solutions at 4 wt % have a Brookfield viscosity of from about 20 to about 1000 mPa*s, at 20° C. and 20 rpm in 100 pbw of a mixture of water and alcohol containing from about 30 to about 60 wt % of alcohol to form a dispersion;
b. heating the dispersion at a temperature of from about 35 to about 55° C. and adding from about 0.5 to about 10 pbw of a cellulase preparation for each 100 pbw of carboxymethylcellulose to the dispersion, and stirring the dispersion at this temperature for from about 60 to about 200 minutes;
c. removing the alcohol from the mixture by distillation;
d. deactivating the cellulase preparation by alkalinizing and heating the mixture at from about 60 to about 70° C. for from about 20 to about 120 minutes; and
e. cooling the mixture at from about 40 to about 55° C. and adding from about 1 to about 5 pbw of a 30 to 35 wt % aqueous solution of hydrogen peroxide per each 100 pbw of carboxymethylcellulose to the dispersion and stirring the dispersion at from about 55 to about 70° C. for from about 15 to about 45 minutes to form an aqueous solution of carboxymethylcellulose.

27. The aqueous solution of carboxymethylcellulose of claim 26, wherein the aqueous solution of carboxymethylcellulose is stable.

28. The aqueous solution of carboxymethylcellulose of claim 26, wherein the carboxymethylcellulose which is dispersed in step a. has a degree of substitution between about 0.6 and about 0.8.

29. The aqueous solution of carboxymethylcellulose of claim 26, wherein the carboxymethylcellulose which is dispersed in step a. has a Brookfield viscosity at 4 wt % of between about 20 and about 500 mPa*s, at 20° C. and 20 rpm.
